(19) United States
(12) Patent Application Publication
(10) Pub. No.: US 2026/0120829 A1
(43) Pub. Date: Apr. 30, 2026

(54) METHOD FOR GENERATING INFORMATION, MEDICAL APPARATUS, AND NON-TRANSITORY COMPUTER-READABLE MEDIUM

(71) Applicant: GE Precision Healthcare LLC, Waukesha, WI (US)

(72) Inventors: Minyu Gu, Wuxi (CN); Ke Tao, Wuxi (CN); Zhiwen Wang, Wuxi (CN)

(21) Appl. No.: 19/374,976

(22) Filed: Oct. 30, 2025

(30) Foreign Application Priority Data
Oct. 31, 2024 (CN) 202411544994.3

(51) Int. Cl. G16H 10/60; G06F 3/04817; G16H 15/00

(52) U.S. Cl. CPC G16H 10/60; G06F 3/04817; G16H 15/00

(57) ABSTRACT

A method for generating information includes: receiving electronic data of a first medical examination report; analyzing content of the first medical examination report based on the electronic data; and generating report information based on a result of the analysis, the report information being used to generate a second medical examination report. The present application can reduce manual edit operations on a template or an examination protocol of a medical examination report by a physician, thereby improving work efficiency.

### CROSS-REFERENCE TO RELATED APPLICATION

This application claim priority to Chinese Patent Application No. 202411544994.3, which was file on October 31, 2024 at the Chinese Patent Office. The entire contents of the above-listed application are incorporated by reference herein in their entirety.

### TECHNICAL FIELD

Embodiments of the present application relate to the field of information processing technologies, and in particular, to a method for generating information, a medical apparatus, and a non-transitory computer-readable medium.

### BACKGROUND

Medical imaging devices can non-invasively obtain internal tissue images of an object to be imaged. For example, a scanning device of the medical imaging device may scan a predetermined site of the object to be imaged to obtain imaging data including information about the predetermined site.

Common medical imaging devices are, for example, ultrasound imaging systems, magnetic resonance imaging (MRI) systems, computed tomography (CT) scanning systems, etc.

After the medical imaging device scans the subject to be imaged, a physician fills out a medical examination report based on information related to the scan. The medical examination report has a predetermined format to meet predetermined requirements.

It should be noted that the above introduction of the background is only for the convenience of clearly and completely describing the technical solutions of the present application, and for the convenience of understanding for those skilled in the art.

### SUMMARY OF THE INVENTION

In some cases, information for generating a medical examination report needs to be updated, the information being, for example, a template of the medical examination report or an examination protocol (for example, a scan protocol) of a medical examination to which the medical examination report relates.

For example, some hospitals have specific templates for medical examination reports, and thus a default template of a medical examination report in a medical imaging device cannot be directly used. For another example, due to an update of an examination protocol of a medical imaging device, or a change of actual requirements, the template of the medical examination report must be updated accordingly. Although there are tools for designing a template of a medical examination report in the prior art, these tools still require a physician to manually make or modify the template of the medical examination report; not only is this time-consuming, but it is also difficult to accurately obtain a required template. In addition, the physician also needs to manually set or edit the examination protocol to which the medical examination report relates, and thus the efficiency of generating or updating the examination protocol is low.

In order to resolve at least one technical problem described above or a similar technical problem, embodiments of the present application provide a method for generating information, a medical apparatus, and a non-transitory computer-readable medium. In the method for generating information, report information is generated based on an analysis result of a first medical examination report and used to generate a second medical examination report. Thus, the physician does not need to manually edit the template or examination protocol of the medical examination report, improving work efficiency.

According to an aspect of the embodiments of the present application, a method for generating information is provided. The method comprises:

Receiving electronic data of a first medical examination report;

Analyzing content of the first medical examination report based on the electronic data; and

generating report information based on a result of the analysis, the report information being used to generate a second medical examination report.

According to another aspect of the embodiments of the present application, a medical apparatus is provided. The medical apparatus comprises:

A receiving unit that receives electronic data of a first medical examination report; and

A processing unit that executes the method for generating information as described in the above embodiments.

According to yet another aspect of the embodiments of the present application, a non-transitory computer-readable medium is provided. The non-transitory computer-readable medium stores a computer program, the computer program, when executed by a computer, causing the computer to execute the steps of the method as described in the above embodiments.

One of the beneficial effects of the embodiments of the present application is that: In the method for generating information, report information is generated based on an analysis result of a first medical examination report and used to generate a second medical examination report. Thus, the physician does not need to manually edit the template or examination protocol of the medical examination report, improving work efficiency.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may

obtain other implementations according to the drawings without involving inventive effort. In the drawings:

FIG. 1 is a schematic diagram of a method for generating information according to some embodiments of the present application;

FIG. 2 is a schematic diagram of a first medical examination report according to an embodiment of the present application;

FIG. 3 is a schematic diagram of a method for analyzing content of a first medical examination report;

FIG. 4 is a schematic diagram of an examination report template generated in operation 103;

FIG. 5 is a schematic diagram of generating an examination protocol according to an embodiment of the present application;

FIG. 6 is a schematic diagram of a user interface generated in operation 104;

FIG. 7 is a schematic diagram of a medical apparatus according to an embodiment of the present application; and

FIG. 8 is a schematic diagram of an ultrasound imaging system according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc. are used to distinguish between different elements in terms of appellation, but do not represent a spatial arrangement, a temporal order, or the like of these elements, and these elements should not be limited by these terms. The term “and/or” includes any one of and all combinations of one or more associated listed terms. The terms “include”, “comprise”, “have”, etc. refer to the presence of described features, elements, components, or assemblies, but do not exclude the presence or addition of one or more other features, elements, components, or assemblies. The terms “pixel” and “voxel” may be used interchangeably.

In the embodiments of the present application, the singular forms “a”, “the” and the like, include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to...” and the term “based on” should be construed as “based at least in part on...”, unless otherwise specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

Some embodiments of the present application provide a method for generating information.

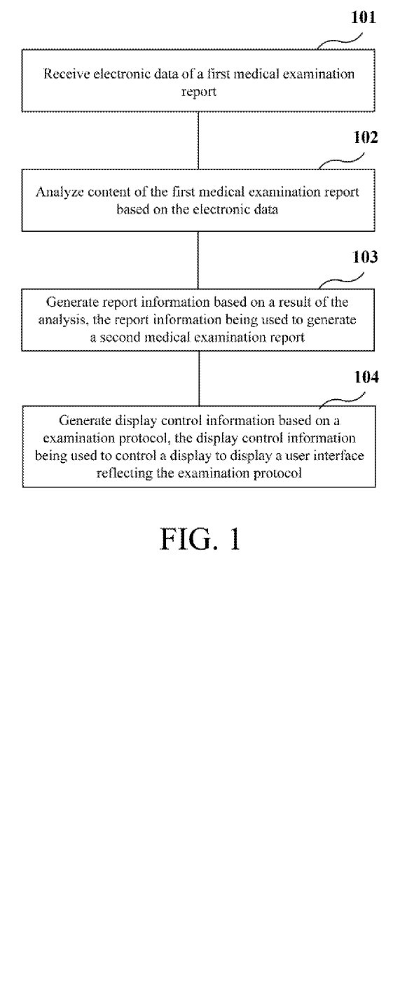

FIG. 1 is a schematic diagram of a method for generating information according to some embodiments of the present application. As shown in FIG. 1, the method for generating information includes:

Operation 101: receiving electronic data of a first medical examination report;

Operation 102: analyzing content of the first medical examination report based on the electronic data; and

Operation 103: generating report information based on a result of the analysis, the report information being used to generate a second medical examination report.

In the present application, the report information generated by using the above method for generating information can be used to generate a second medical examination report. The report information may include at least one of the following pieces of information: an examination protocol of a medical examination to which the second medical examination report relates; and an examination report template for generating the second medical examination report.

A type of the medical examination in the second medical examination report is the same as a type of the medical examination in the first medical examination report. For example, both are ultrasound examinations performed on a specific site (for example, a thyroid) of a human body.

According to the embodiments of the present application, report information is generated based on the analysis result of the first medical examination report, and used to generate the second medical examination report. Therefore, the physician does not need to perform manual input on the template or examination protocol of the medical examination report, improving work efficiency.

In addition, in the present application, the physician can edit the generated template and/or examination protocol of the medical examination report as needed, thereby improving flexibility.

In the present application, the method for generating information shown in FIG. 1 can be applied to a medical examination device, which may be a medical imaging device, such as an ultrasound imaging device, a magnetic resonance imaging (MRI) device, or a computed tomography (CT) imaging device. In addition, the medical examination device may alternatively be of another type, for example, an electrocardiogram examination device.

The first medical examination report may be an examination report of medical imaging, for example, the medical imaging may be ultrasound imaging, magnetic resonance imaging (MRI), or computed tomography (CT) imaging. In addition, the first medical

examination report may also be another type of medical examination report, for example, an electrocardiogram examination report.

In some embodiments of operation 101, the first medical examination report may be a paper-based file. Thus, the electronic data of the first medical examination report may include image data obtained by scanning or photographing the paper-based first medical examination report, a format of the image data being, for example, a picture format such as tagged image file format (tiff) or joint photographic experts group (JPEG), or a document format such as portable document format (pdf) or Word. In some examples, a scanner or a camera scans or photographs the paper-based first medical examination report to obtain image data, and transmits the image data to the medical examination device by wired or wireless means. In some other examples, the image data of the first medical examination report is stored in a storage device (for example, a mobile hard disk or a USB flash drive), and the storage device is connected to the medical examination device, so that the image data is input into the medical examination device.

In some other embodiments of operation 101, the first medical examination report may be an electronic file, which is an editable or non-editable file. The electronic data of the electronic file is, for example, document data, and a format of the electronic file is, for example, a document format such as portable document format (pdf) or Word. In some other examples, the electronic data of the first medical examination report is stored in a storage device (for example, a mobile hard disk or a USB flash drive), and the storage device is connected to the medical examination device, so that the image data is input into the medical examination device.

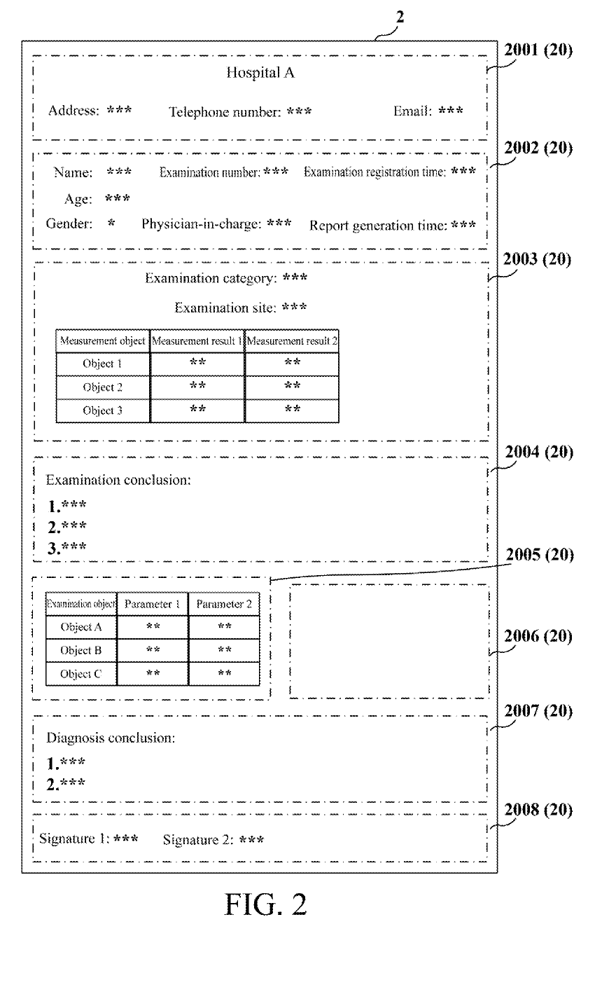

FIG. 2 is a schematic diagram of a first medical examination report according to an embodiment of the present application. As shown in FIG. 2, each information region 20 of a first medical examination report 2 has been filled with corresponding content, and a format of the content (that is, a content format) may be at least one of text, image, table, and graph. The text may include letters, Chinese characters or characters in other languages (for example, Japanese or Korean) and numbers; the image may be a medical image, for example, an ultrasound image; the table may include one or more rows (columns) of cells, and each cell may be filled with content such as a character or a number; and the graph is, for example, in a form of a line graph, a bar graph, or a pie chart.

The content in each information region 20 of the first medical examination report 2 may be content written by the physician with a pen, content input by means of an input device such as a keyboard, or content generated by the medical examination device (for example, at least one of a medical image, a table, and a graph generated by the medical examination device).

As shown in FIG. 2, the first medical examination report 2 includes a plurality of information regions 20. For example, the plurality of information regions 20 may be labeled as 2001 to 2008. The content of each information region 20 is as follows:

Information region 2001: for example, this information region may have a name of a medical institution, a logo of the medical institution, and contact information of the medical institution(for example, at least one of information such as an address, a contact phone number, an email address, a website, a two-dimensional code, and an official account), etc.;

Information region 2002: for example, this information region may include at least one piece of information such as a name, an age, a gender, an examination number, a physician-in-charge, an examination registration time, and a generation time of a medical examination report of a subject to be examined;

Information region 2003: for example, this information region may include at least one piece of information such as a name of an examination type (for example, color Doppler), an examination site, and a measurement result obtained during the examination (for example, a measurement result obtained by measuring a medical image obtained during a medical examination), wherein the measurement result may be in table form;

Information region 2004: for example, this information region may include an objective description of an examination result;

Information region 2005: for example, this information region may include physiological parameters of the subject to be examined obtained during the examination, such as a resistance index (RI) and a pulsatility index (PI) of middle cerebral artery blood flow, wherein various physiological parameters are listed in table form;

Information region 2006: for example, this information region may include a medical image obtained during the medical examination, and the medical image may include at least one of a medical picture (for example, an ultrasound image), a waveform, a curve, and a graph (for example, a bar graph);

Information region 2007: for example, this information region may include a diagnosis of the physician who performed the medical examination; and

Information region 2008: for example, this information region may include a signature of the physician and/or other personnel who performed the medical examination.

FIG. 2 shows an example of the information regions 20 of the first medical examination report 2, but the present application is not limited thereto, and each information region 20 of the first medical examination report 2 may have other content.

In operation 102, content of the first medical examination report is analyzed based on the electronic data of the first medical examination report received in operation 101. In some examples, an artificial intelligence model or other types of models may be used to analyze the electronic data to obtain a result of the analysis. For example, an artificial intelligence model may be used to perform optical character recognition (OCR) on at least one information region 20 in the first medical examination report 2, thereby recognizing text in the information region 20, and then determining a type of the first medical examination in the first medical examination report 2 based on the recognized text.

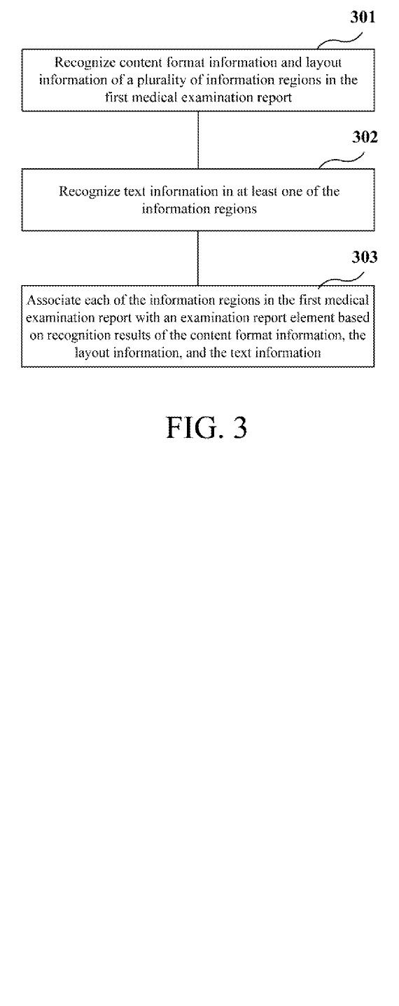

FIG. 3 is a schematic diagram of a method for analyzing content of a first medical examination report. The method for analyzing the content of the first medical examination report is an example for implementing operation 102. As shown in FIG. 3, the method for analyzing the content of the first medical examination report includes:

Operation 301: recognizing content format information and layout information of a plurality of information regions in the first medical examination report;

Operation 302: recognizing text information in at least one of the information regions; and

Operation 303: associating each of the information regions in the first medical examination report with an examination report element based on recognized results of the content format information, the layout information, and the text information.

Operation 301 may also be referred to as structure analysis. In some embodiments of operation 301, an artificial intelligence (AI) model may be used to recognize the content format information and the layout information of each information region 20 in the first medical examination report 2.

The content format information, for example, is that the content of each information region 20 in the first medical examination report 2 is at least one of text, image, table, and graph, or is in another format.

The layout information may be information such as a position and a size of each information region 20 in the first medical examination report 2. For example, a coordinate system may be set by using a vertex at a lower left corner of the first medical examination report 2 as an origin O and using a width (W) direction and a height (H) direction of the first medical examination report 2 as directions of two orthogonal coordinate axes; a position of a geometric center point or a position of a predetermined of each information region 20 may be used as a position of the information region 20 vertex (for example, an information region 20 being a rectangle, and a vertex at an upper left corner of the rectangle being used as the predetermined vertex). In addition, the size of the information region 20 may be the size of the information region 20 in the width (W) direction and the size of the information region 20 in the height (H) direction.

In addition, the present application is not limited thereto, and operation 301 may also use other models other than an artificial intelligence model, a combination of an artificial intelligence model and another model, or the like.

In operation 302, an artificial intelligence model or another type of model may be used to perform optical character recognition (OCR) on an information region 20 containing text, thereby recognizing text information in the information region 20. A type of the information region 20 may be determined by means of the text in the information region 20.

In operation 303, each information region 20 in the first medical examination report 2 can be associated with an examination report element based on the recognition results in operation 301 and operation 302 (for example, the recognition results of the content format information, the layout information, and the text information of each information region 20 in the first medical examination report 2).

The examination report element is a component of an examination report template. The examination report element may be, for example, at least one of information about a medical institution, information about a subject to be examined, an examination type, an examination conclusion, an examination parameter, a medical examination image, an impression, and a signature. The examination report element may be an examination report element stored in the medical examination device, or a newly generated examination report element.

In some embodiments of operation 303, an artificial intelligence model or another model may be used to associate each information region 20 in the first medical examination report 2 with an existing examination report element (for example, the existing examination report element refers to an examination report element stored in the medical examination device). For example, the recognition results (for example, the recognition results of the content format information, the layout information, and the text information) of each information region 20 in the first medical examination report 2 are input into an artificial intelligence model, and the artificial intelligence model outputs a correspondence relationship between each information region 20 and an examination report element. For example, the correspondence relationship may be that:

An examination report element corresponding to information region 2001 is the “information about a medical institution”;

An examination report element corresponding to information region 2002 is the “information about a subject to be examined”;

An examination report element corresponding to information region 2003 is the “examination type”;

An examination report element corresponding to information region 2004 is the “examination conclusion”;

An examination report element corresponding to information region 2005 is the “examination parameter”;

An examination report element corresponding to information region 2006 is the “medical examination image”;

An examination report element corresponding to information region 2007 is the “impression”; and

An examination report element corresponding to information region 2008 is the “signature”.

In addition, in operation 303, if an information region 20 in the first medical examination report 2 cannot be associated with an existing examination report element, a new examination report element can be generated for the information region 20, and the information region 20 associated with the generated new examination report element. In addition, the generated new examination report element can be stored in the medical examination device, thereby enriching the types of examination report elements stored in the medical examination device.

In operation 103, an examination report template can be generated as the report information based on the result of association obtained in operation 303 between each information region 20 in the first medical examination report 2 and an examination report element. For example, a position, a size, and text of a corresponding region in the examination report template are set based on position information, size information, and text information of each information region 20 in the first medical examination report 2, and an attribute of a corresponding region in the examination report template is set based on the examination report element associated with the information region 20. The attribute of the corresponding region may be at least one of the following examples:

Text and/or a table in the region is editable; graphics and/or text in the region is fixed and cannot be edited; and a medical image generated by the medical examination device is received and displayed in the region.

Furthermore, in a process of generating the examination report template in operation 103, information in at least one information region 20 in the first medical examination report 2 can be set to predetermined information, that is, information in at least one information region 20 in the first medical examination report 2 is modified or replaced with predetermined information.

For example, information about a medical institution in information region 2001 of the first medical examination report 2 is information about medical institution A, and the

examination report element associated with information region 2001 is set to have information about medical institution B, and thus the generated examination report template has information about medical institution B in a region corresponding to information region 2001.

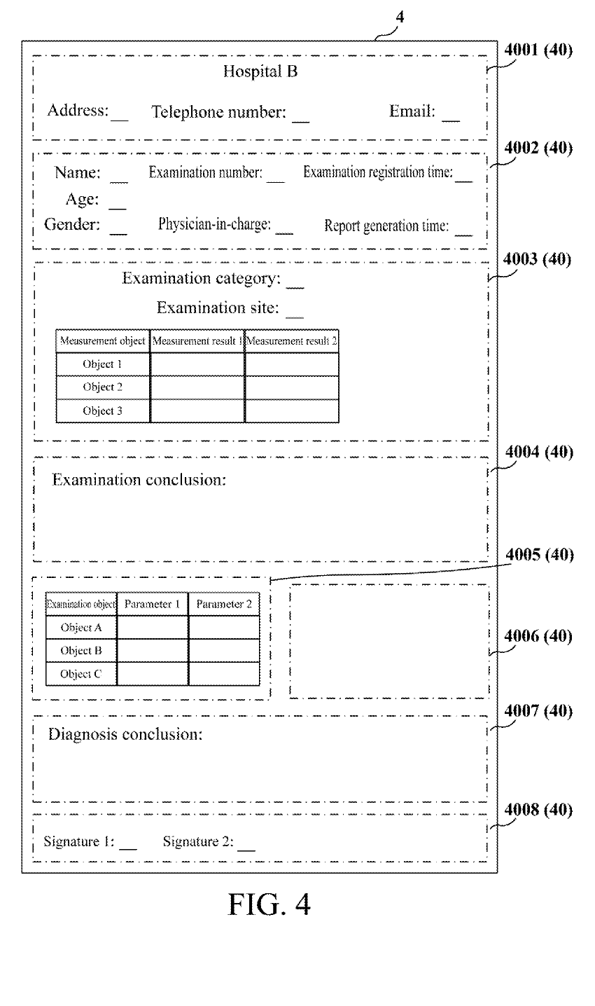

FIG. 4 is a schematic diagram of an examination report template generated in operation 103. As shown in FIG. 4, an examination report template 4 may have a plurality of template regions 40, for example, template regions 4001 to 4008. Positions and sizes of template regions 4001 to 4008 may correspond to information regions 2001 to 2008, respectively, in the first medical examination report 2 in FIG. 2. Attributes of template regions 4001 to 4008 may be set based on attributes of corresponding examination report elements, respectively.

In some embodiments of the present application, the examination report template 4 generated in operation 103 can be stored in the medical examination device. For example, the examination report template 4 generated in operation 103 may be in hypertext markup language (html) format or extensible markup language (xml) format, or another format.

The examination report template 4 may be used to generate the second medical examination report. For example, when an examination report template 4 is generated, after examining the subject to be examined by using the medical examination device, the physician can use the examination report template 4 to generate a medical examination report (that is, the second medical examination report).

In some embodiments of the present application, the report information generated in operation 103 is an examination report template, and thus, by means of the method in the present application, the physician can quickly generate the examination report template 4 by using the first medical examination report 2 in a paper-based or electronic version. The method in the present application is more efficient than a method of drawing a template by using an editor. Moreover, information such as a position, a size and a type of each template region 40 in the examination report template 4 generated by using the method in the present application is the same as those in information regions 2001 to 2008 in the first medical examination report 2, thereby improving accuracy of the examination report template 4. According to the embodiments of the present application, when the first medical examination report 2 is, for example, a medical examination report that meets the latest requirements of the medical examination device or the examination protocol, the physician can quickly and accurately generate the examination report template 4 corresponding to the first medical examination report 2 by means of the method in the present application, thereby quickly updating the examination report template.

In some other embodiments of the present application, the report information generated in operation 103 may include an examination protocol of a medical examination to which the second medical examination report relates. For example, the report information generated in operation 103 may include only an examination protocol, or include both an examination protocol and an examination report template, or include only an examination report template.

In the present application, an examination protocol may also be referred to as an examination scheme, and an examination protocol may include an examination type, an examination site (for example, a body site or an organ of a human body), an examination sequence, a parameter that needs to be measured in the examination, and the like. When a medical examination involves scanning (for example, ultrasound scanning, X-ray scanning, or magnetic resonance scanning), the examination protocol may be referred to as a scan protocol.

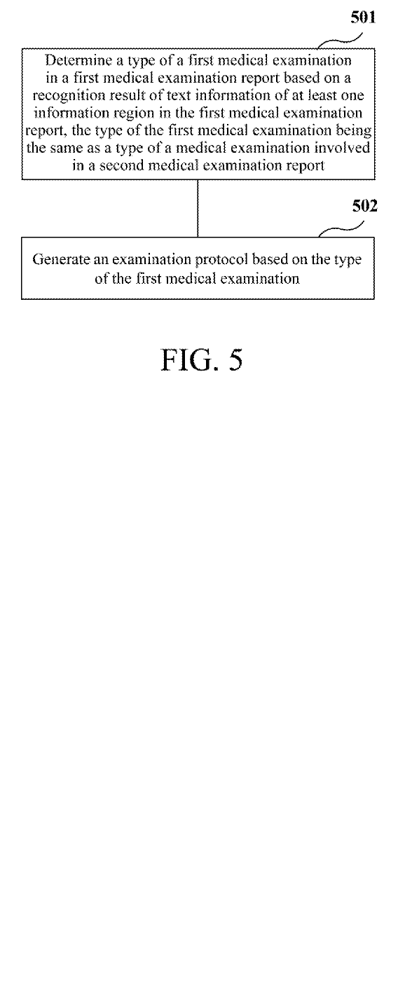

FIG. 5 is a schematic diagram of generating an examination protocol according to an embodiment of the present application, serving as an embodiment of operation 103. As shown in FIG. 5, a method for generating an examination protocol may include:

Operation 501: determining a type of a first medical examination in the first medical examination report based on a recognition result of text information of at least one information region in the first medical examination report, the type of the first medical examination being the same as a type of a medical examination involved in the second medical examination report; and

Operation 502: generating the examination protocol based on the type of the first medical examination.

In the present application, a type of a medical examination in a medical examination report or a type of a medical examination to which a medical examination report relates means that: the physician performs that type of medical examination on a subject to be examined, thereby generating the medical examination report.

In some embodiments of operation 501, the type of the first medical examination in the first medical examination report 2 can be determined based on the recognition result of the text information of at least one information region 20 in the first medical examination report 2.

For example, at least one of information such as a name of an examination type, an examination site, and a measurement result obtained during the examination can be determined based on the recognition result of the text information in information region 2003 of FIG. 2 (for example, based on the recognition result of operation 302). Then, the type of the first medical examination involved in the first medical examination report 2 is determined based on a correspondence relationship between the recognition result of the text information and the type of the medical examination (for example, based on a lookup table or by using an artificial intelligence model), and the type of the first medical examination is the same as the type of the medical examination designed by the second medical examination report.

It should be noted that, when the report information generated in operation 103 includes only the examination protocol, in some examples, operation 102 may include only operation 302, and not include operation 301 and operation 303, that is, in operation 102, the text information of the information region is recognized by using operation 302, and the recognition result of the text information is used in operation 103 to generate the examination protocol.

In operation 502, an examination protocol is generated based on the type of the first medical examination determined in operation 501. For example, a correspondence relationship between the type of the medical examination and the examination protocol (for example, the correspondence relationship being in the form of a lookup table) is stored in the medical examination device. Thus, an examination protocol corresponding to the type of the first medical examination is determined based on the correspondence relationship according to the type of the first medical examination.

The examination protocol generated in operation 502 may be a configuration file that may list at least one of an examination mode (for example, a scan mode), an examination plane (for example, a scan plane), and a measurement parameter required for a medical examination (for example, a scan). In addition, an order of predetermined examination steps (for example, an order of scanning steps) and the like may be set in the configuration file.

Information such as an examination step, an examination mode, an examination plane, and a measurement parameter set in the examination protocol can be displayed on a user interface (UI), so as to guide the physician to perform a medical examination based on the examination protocol. Thus, a burden on the physician can be reduced and a medical examination process is made to be more reliable.

As shown in FIG. 1, the method for generating information further includes:

Operation 104: generating display control information based on the examination protocol, the display control information being used to control a display to display a user interface reflecting the examination protocol.

In some embodiments of operation 104, the user interface may include a first icon. The first icon is used to represent each examination step in the examination protocol. For example, a plurality of first icons are displayed in the user interface, each first icon corresponding to one examination step, and shapes of the first icons may be the same or different. Thus, the physician can easily determine the number of examination steps in the examination protocol by means of observing the number of first icons.

In some examples, a display state of the first icon may correspond to a completion state of the examination step. The display state of the first icon may include: at least one of a color, a grayscale, a brightness, a transparency, and a filled texture of the first icon; and/or a blinking frequency of the first icon; and/or the size of the first icon. The completion state of the examination step, for example, includes the following states: the examination step has not been performed, the examination step is currently being performed, the examination step has been completed, the examination step has failed, or the like.

For example, when a specific examination step has not been performed, the first icon corresponding to the examination step has a first display state; when the examination step is currently being performed, the first icon corresponding to the examination step has a second display state; and after a specific examination step is completed, the first icon corresponding to the examination step has a third display state. The first display state, the second display state, and the third display state may differ in at least one of: the color of the first icon, the grayscale of the first icon, the brightness of the first icon, the transparency of the first icon, the filled texture of the first icon, the blinking frequency of the first icon, and the size of the first icon.

Thus, the display state of the first icon can indicate the completion state of the corresponding examination step, and the physician can easily determine a completion state of each examination step in the examination protocol by means of observing the display state of the first icon.

In some embodiments of operation 104, the user interface may further include at least one of the following pieces of information:

A schematic diagram of an examination object of at least one examination step, where for example, the examination object of the examination step is a transverse cross-section of a thyroid, and a schematic diagram of the transverse cross-section of the thyroid may be displayed near the first icon of the examination step (for example, below the first icon);

A medical image obtained in the at least one examination step, where for example, the examination step is to perform an ultrasound scan on the transverse cross-section of the thyroid, and an ultrasound image obtained by performing the ultrasound scan on the transverse cross-section of the thyroid may be displayed on the user interface; and

An operation menu of the at least one examination step, where the operation menu may include a list of options corresponding to operations such as measurement, recognition of a target region, or drawing a contour of the target region; the physician performs an operation on an option in the operation menu, the medical examination device can perform corresponding processing, and a processing result can be displayed on the user interface; for example, the examination step is to measure the size of a nodule, the operation menu of the examination step is displayed near the first icon (for example, below the first icon) corresponding to the examination step, and the list of options of the operation menu may include: length (L), height (H), width (W), automatically drawing a contour on a height and length plane (Auto Contour (H*L)), and automatically drawing a contour on a height and width plane (Auto Contour (H*W)), where the medical examination device can perform corresponding processing when the physician selects a specific option, and the processing result can be displayed on the user interface.

In addition, the user interface may further display other information, which is not limited in the present application.

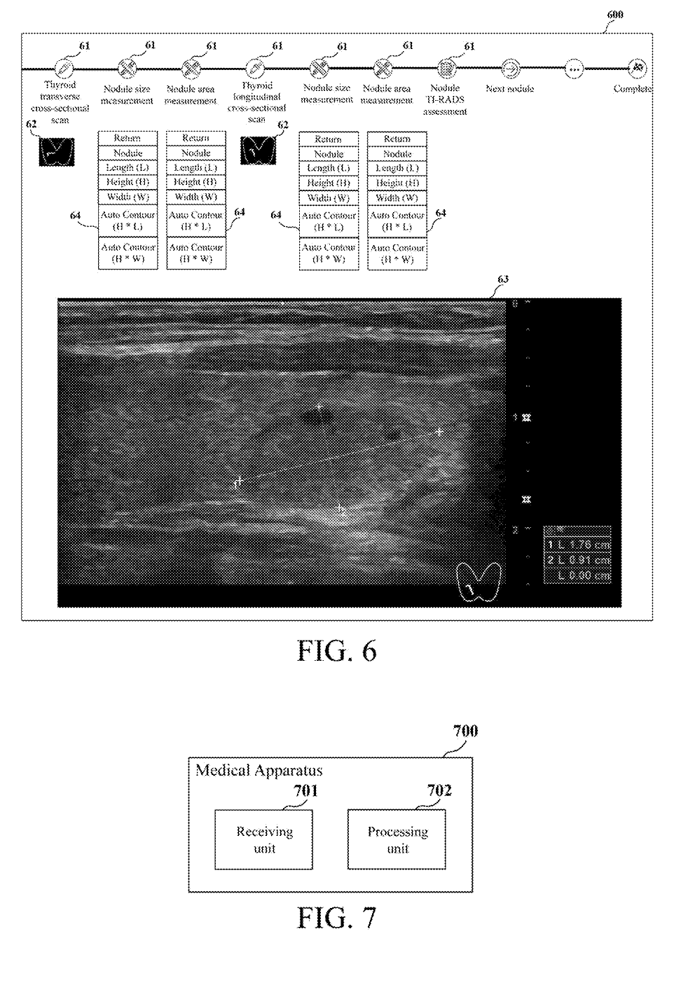

FIG. 6 is a schematic diagram of a user interface generated in operation 104. FIG. 6 is described using an example in which a thyroid of a subject to be examined is scanned by using an ultrasound device, that is, the type of the first medical examination is an ultrasound scan on the thyroid of the subject to be examined.

As shown in FIG. 6, the user interface 600 may include a plurality of first icons 61, and shapes of the first icons 61 may be the same or different. The plurality of first icons 61 are arranged in a row along the horizontal direction of the user interface 600. The plurality of first icons 61 represent corresponding examination steps in the examination protocol, respectively, and the name of the examination step is displayed near each first icon 61 (for example, below each first icon 61). For example, names of the examination steps may be: thyroid transverse cross-sectional scan, nodule size measurement, nodule area measurement, thyroid longitudinal cross-sectional scan, nodule size measurement, nodule area measurement, nodule TI-RADS assessment, and next nodule. In addition, the examination protocol may further include other examination steps not shown in the figure.

As shown in FIG. 6, when a specific examination step has not been performed, the first icon 61 corresponding to the examination step has a first display state; when the examination step is currently being performed, the first icon 61 corresponding to the examination step has a second display state; and after a specific examination step is completed, the first icon 61 corresponding to the examination step has a third display state. The first display state, the second display state, and the third display state may differ in at least one of: a color of the first icon 61, a grayscale of the first icon 61, a brightness of the first icon 61, a transparency of the first icon 61, a filled texture of the first icon 61, a blinking frequency of the first icon 61, and a size of the first icon 61.

For example, in the first display state, the first icon 61 is amber; in the second display state, the first icon 61 is yellow; and in the third display state, the first icon 61 is green.

After an examination step is completed, the display state of the first icon 61 corresponding to the examination step is switched from the second display state to the third display state, and the display state of the first icon 61 corresponding to a next examination step is switched from the first display state to the second display state; if the next examination step has an operation menu 64, the operation menu 64 is activated (that is, options in the operation menu 64 can be selected). Thus, by means of a change of the display state of the first icon 61, the physician is guided to perform the medical examination based on an examination order set in the examination protocol.

As shown in FIG. 6, the user interface 600 may further include a schematic diagram 62 of an examination object of at least one examination step. For example, for the examination step of the thyroid transverse cross-sectional scan, the examination object is a transverse cross-section of the thyroid, and a schematic diagram of the transverse cross-section of the thyroid can be displayed as the schematic diagram 62 of the examination object near the first icon 61 (for example, below the first icon 61) of the examination step. For another example, for the examination step of the thyroid longitudinal cross-sectional scan, the examination object is a longitudinal cross-section of the thyroid, and a schematic diagram of the longitudinal cross-section of the thyroid can be displayed near the first icon 61 (for example, below the first icon 61) of the examination step as the schematic diagram 62 of the examination object.

As shown in FIG. 6, the user interface 600 may further include a medical image 63 obtained in the at least one examination step. For example, the examination step is to measure the size of the nodule in the longitudinal cross-section of the thyroid, and an ultrasound image obtained by performing an ultrasound scan on the transverse cross-section of the thyroid that is displayed on the user interface 600 can be used as the medical image 63, and a line segment 1 for representing the size of the nodule in the length direction (L) and a line segment 2 for representing the size of the nodule in the height direction (H) are displayed on the medical image 63.

As shown in FIG. 6, the user interface 600 may further include an operation menu 64 of the at least one examination step. The operation menu may include a list of options corresponding to operations such as measurement, recognition of a target region, or drawing a contour of the target region; the physician performs an operation on an option in the operation menu, the medical examination device can perform corresponding processing, and the processed result can be displayed on the user interface.

For example, when the examination step is to measure the size of the nodule in the longitudinal cross-section of the thyroid, the operation menu 64 of the examination step is displayed near the first icon 61 (for example, below the first icon) corresponding to the examination step, and the list of options of the operation menu 64 may include: length (L), height (H), width (W), automatically drawing a contour on a height and length plane (Auto Contour (H*L)), and automatically drawing a contour on a height and width plane (Auto Contour (H*W)). The medical examination device can perform corresponding processing when the physician selects a specific option, and the processing result can be displayed on the user interface 600. For example, if the physician selects a length (L) option, the physician can mark the line segment 1 for representing the size of the nodule in the length direction (L) on the medical image 63 by using a mouse or other tool, and the size of the nodule corresponding to the line segment 1 is displayed near the medical image 63 (for example, displayed in a list 65).

Some embodiments of the present application further provide a medical apparatus.

FIG. 7 is a schematic diagram of a medical apparatus according to an embodiment of the present application.

As shown in FIG. 7, a medical apparatus 700 includes:

A receiving unit 701 that receives electronic data of a first medical examination report; and

A processing unit 702 that performs the method for generating information as shown in FIG. 1.

In some embodiments, the processing unit 702 analyzes content of the first medical examination report based on the electronic data, and generates report information based on a result of the analysis, the report information being used to generate a second medical examination report.

In some embodiments, the report information includes an examination protocol of a medical examination to which the second medical examination report relates. Generating report information based on a result of the analysis includes:

Determining a type of a first medical examination in the first medical examination report based on a recognition result of text information of at least one information region in the first medical examination report, the type of the first medical examination being the same as a type of the medical examination to which the second medical examination report relates; and

Generating the examination protocol based on the type of the first medical examination.

In some embodiments, the processing unit 702 may further generate display control information based on the examination protocol, the display control information being used to control a display to display a user interface reflecting the examination protocol.

In some examples, the user interface includes: a first icon for representing each examination step in the examination protocol, a display state of a first icon corresponding to a completion state of the examination step.

In some examples, the user interface further includes at least one of the following pieces of information:

A schematic diagram of an examination subject of at least one examination step;

A medical image obtained in the at least one examination step; and

An operation menu of the at least one examination step.

In some embodiments, the report information further includes an examination report template for generating the second medical examination report. Analyzing content of the first medical examination report based on the electronic data includes:

Recognizing content format information and layout information of a plurality of information regions in the first medical examination report;

Recognizing text information in at least one of the information regions; and

Associating each of the information regions in the first medical examination report with an examination report element based on recognition results of the content format information, the layout information, and the text information.

In some examples, an examination report element is generated for at least one of the information regions in the first medical examination report, and the information region is made to be associated with the generated examination report element.

In some examples, generating report information based on a result of the analysis includes:

generating the examination report template based on a result of the association.

In some examples, in the examination report template, information in the at least one of the information regions in the first medical examination report is set as predetermined information.

For further description of each unit in the medical apparatus 700, reference may be made to a detailed description of a corresponding operation in FIG. 1.

It can be understood that the medical apparatus 700 may include different types, and for example, the medical apparatus 700 may be a medical imaging system, such as an ultrasound imaging system, a magnetic resonance imaging (MRI) system, or a computed tomography (CT) imaging system. The description below uses the medical apparatus 700 being an ultrasound imaging system as an example.

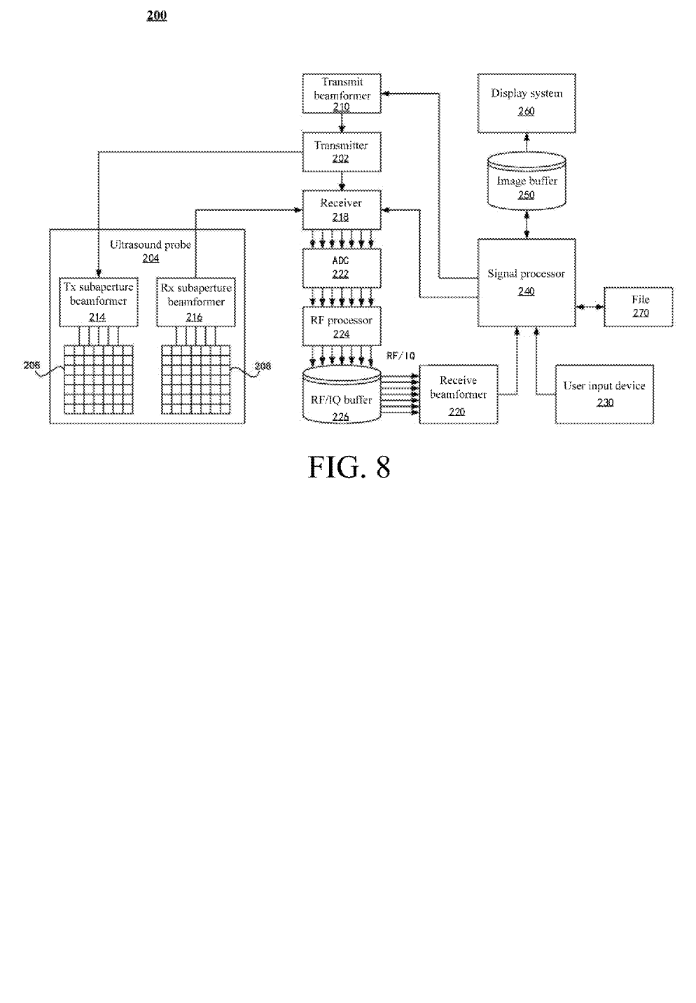

FIG. 8 is a schematic diagram of an ultrasound imaging system according to an embodiment of the present application. As shown in FIG. 8, the ultrasound imaging system 200 may be configured to provide ultrasound imaging, and thus may include suitable circuitry, interfaces, logic, and/or code for performing and/or supporting ultrasound imaging-related functions.

The ultrasound imaging system 200 includes, for example, a transmitter 202, an ultrasound probe 204, a transmit beamformer 210, a receiver 218, a receive beamformer 220, an RF processor 224, an RF/IQ buffer 226, a receiving unit (shown in the figure by using a user input device 230 as an example), a signal processor 240 (corresponding to the processing unit 702), an image buffer 250, a display system 260 (a display), and a file 270.

The transmitter 202 may include suitable circuitry, interfaces, logic, and/or code operable to drive the ultrasound probe 204. The ultrasound probe 204 may include an array of two-dimensional (2D) piezoelectric elements. The ultrasound probe 204 may include a set of transmitting transducer elements 206 and a set of receiving transducer elements 208 that typically form the same element. In some embodiments, the ultrasound probe 204 is operable to acquire ultrasound image data covering at least a substantial portion of an anatomical structure (such as the heart or any suitable anatomical structure).

The transmit beamformer 210 may include suitable circuitry, interfaces, logic, and/or code that is operable to control the transmitter 202, and the transmitter 202 drives the set of transmitting transducer elements 206 by means of a transmit subaperture beamformer 214 to transmit an ultrasound emission signal into a region of interest (e.g., a person, animal, subsurface cavity, physical structure, etc.). The emitted ultrasound signal can be backscattered from structures in an object of interest (e.g., blood cells or tissue) to produce an echo. The echo is received by the receiving transducer element 208.

The set of receiving transducer elements 208 in the ultrasound probe 204 is operable to convert the received echo to an analog signal for subaperture beam formation through a receiving subaperture beamformer 216, which is then transmitted to the receiver 218. The receiver 218 may include suitable circuitry, interfaces, logic, and/or code that is operable to receive signals from the receiving subaperture beamformer 216. The analog signal can be transferred to one or more of a plurality of A/D converters 222.

The plurality of A/D converters 222 may include suitable circuitry, interfaces, logic, and/or code that is operable to convert the analog signal from the receiver 218 to a corresponding digital signal. The plurality of A/D converters 222 are provided between the receiver 218 and the RF processor 224. Nevertheless, the present application is not limited in this regard. Thus, in some embodiments, the plurality of A/D converters 222 may be integrated within the receiver 218.

The RF processor 224 may include suitable circuitry, interfaces, logic, and/or code that is operable to demodulate the digital signals output by the plurality of A/D converters 222. According to one embodiment, the RF processor 224 may include a complex demodulator (not shown) that is operable to demodulate the digital signal to form an I/Q data pair representing the corresponding echo signal. The RF or I/Q signal data can then be transferred to the RF/IQ buffer 226. The RF/IQ buffer 226 may include suitable circuitry, interfaces, logic, and/or code that is operable to provide temporary storage of RF or I/Q signal data generated by the RF processor 224.

The receive beamformer 220 may include suitable circuitry, interfaces, logic, and/or code that may be operable to perform digital beamforming processing to, for example, sum and output a beam summing signal for the delay-channel signals received from the RF processor 224 via the RF/IQ buffer 226. The resulting processed information may be the beam summing signal outputted from the receive beamformer 220 and transmitted to the signal processor 240. According to some embodiments, the receiver 218, the plurality of A/D converters 222, the RF processor 224, and the beamformer 220 may be integrated into a single beamformer which may be digital. In various embodiments, the ultrasound imaging system 200 includes a plurality of receive beamformers 220.

In the embodiment of FIG. 8, the receiving unit is represented by the user input device 230. The user input device 230 can be used to enter patient data, scan parameters, and settings, and select protocols and/or templates to interact with the AI segmentation processor, so as to select tracking targets, etc. In an illustrative embodiment, the user input device 230 is operable to configure, manage, and/or control the operation of one or more components and/or modules in the ultrasound imaging system 200. In this regard, the user input device 230 is operable to configure, manage, and/or control the operation of the transmitter 202, the ultrasound probe 204, the transmit beamformer 210, the receiver 218, the receive beamformer 220, the RF processor 224, the RF/IQ buffer 226, the user input device 230, the signal processor 240, the image buffer 250, the display system 260, and/or the file 270.

For example, user input devices 230 may include buttons, rotary encoders, touch screens, motion tracking, voice recognition, mouse devices, keyboards, trackballs, cameras, data receiving ports (for example, USB interfaces), and/or any other devices capable of receiving user commands. In some embodiments, for example, one or more of the user input devices 230 may be integrated into other components (such as the display system 260 or the ultrasound probe 204). As an example, the user input device 230 may include a touch screen display. As another example, the user input device 230 may include an accelerometer, gyroscope, and/or magnetometer attached to and/or integrated with the probe 204 to provide pose and motion recognition of the probe 204, such as identifying one or more probe compressions against the patient's body, predefined probe movements, or tilt operations, etc. Additionally and/or alternatively, the user input device 230 may include image analysis processing to identify the probe pose by analyzing the captured image data.

In some other embodiments of the ultrasound imaging system 200, the receiving unit may be at least one of a scanner, a camera, and a data receiving port. For example, the receiving unit is a scanner or a camera. The scanner or the camera scans or photographs the paper-based first medical examination report to obtain image data, and the image data is transmitted to the signal processor 240 of the ultrasound imaging system 200 by means of a wired link (for example, a data line) or a wireless link (for example, a wireless communication link such as Bluetooth or wifi). A format of the image data is, for example, a picture format such as tagged image file format (tiff) or joint photographic experts group (JPEG), or a document format such as portable document format (pdf) or Word. For another example, the receiving unit is a data receiving port of the ultrasound imaging system 200, and the data receiving port may be a USB interface, a type-C interface, or the like. An external storage device (for example, a mobile hard disk or a USB flash drive) can transmit the stored electronic data of the first medical examination report to the ultrasound imaging system 200 by means of the data receiving port.

The signal processor 240 may include suitable circuitry, interfaces, logic, and/or code that is operable to process the ultrasound scan data (i.e., summed IQ signals) to generate an ultrasound image for presentation on the display system 260. The signal processor 240 is operable to perform one or more processing operations based on a plurality of selectable ultrasound modalities on the acquired ultrasound scan data. In an illustrative embodiment, the signal processor 240 is operable to perform display processing and/or control processing, etc. As the echo signal is received, the acquired ultrasound scan data can be processed in real-time during the scan session. Additionally or alternatively, the ultrasound scan data may be temporarily stored in the RF/IQ buffer 226 during the scan session and processed in a less real-time manner during online or offline operation. In various embodiments, the processed image data may be presented at the display system 260 and/or may be stored in the file 270. The file 270 can be a local file, a picture archiving and communication system (PACS), or any suitable device for storing images and related information.

The signal processor 240 may be one or more central processing units, microprocessors, microcontrollers, etc. For example, the signal processor 240 may be an integrated component, or may be distributed in various locations. The signal processor 240 may be configured to receive input information from the user input device 230 and/or file 270, generate outputs that may be shown by the display system 260, and manipulate the outputs, etc., in response to the input information from the user input device 230. The signal processor 240 may be capable of executing, for example, any of one or more of the methods and/or one or more sets of instructions discussed herein according to various embodiments.

In some embodiments of the ultrasound imaging system 200, the signal processor 240 can be used to implement the function of the processing unit 702 in FIG. 7, so as to perform the method for generating information described in the foregoing embodiments of the present application. For example, the signal processor 240 analyzes content of the first medical examination report based on the electronic data of the first medical examination report received by the receiving unit, and generates report information for generating a second medical examination report based on a result of the analysis. The report information may include at least one of an examination protocol and an examination report template. For a detailed description of the method for generating information, reference may be made to the related descriptions of the foregoing embodiments.

The ultrasound imaging system 200 may be operated to continuously acquire ultrasound scan data at a frame rate suitable for the imaging situation under consideration. Typical frame rates are in the range of 20 to 220, but can be lower or higher. The acquired ultrasound scan data can be shown on the display system 260 in real-time at a display rate that is the same as the frame rate, or slower, or faster than the frame rate. The image buffer 250 is included to store for processing frames of the acquired ultrasound scanning data that are not scheduled for immediate display. Preferably, the image buffer 250 has sufficient capacity to store frames of ultrasound scan data for at least a few minutes. Frames of ultrasound scan data are stored in such a way that they can be easily retrieved therefrom according to their acquisition sequence or time. The image buffer 250 may be embodied in any known data storage medium.

In some specific embodiments, the signal processor 240 may be configured to perform or otherwise control at least some of the functions performed thereby based on user instructions via the user input device 230. As an example, the user may provide voice commands, probe poses, button presses, etc. to issue specific commands such as controlling aspects of automatic strain measurement and strain ratio calculations, and/or provide or otherwise specify various parameters or settings associated therewith, as described in more detail below.

During operation, the ultrasound imaging system 200 may be used to generate ultrasound images, including two-dimensional (2D), three-dimensional (3D), and/or four-dimensional (4D) images. In this regard, the ultrasound imaging system 200 is operable to continuously acquire ultrasound scan data at a specific frame rate, which may be applicable to the imaging situation discussed. For example, the frame rate can be in the range of 20-70, or can be lower or higher. The acquired ultrasound scanning data can be shown on the display system 260 at the same display rate as the frame rate, or slower or faster than the frame rate. The image buffer 250 is included to store for processing frames of the acquired ultrasound scanning data that are not scheduled for immediate display. Preferably, the image buffer 250 has sufficient capacity to store at least a few seconds of frames of ultrasound scan data. Frames of ultrasound scan data are stored in such a way that they can be easily retrieved therefrom according to their acquisition sequence or time. The image buffer 250 may be embodied in any known data storage medium.

In some cases, the ultrasound imaging system 200 may be configured to support grayscale and color-based operations. For example, the signal processor 240 may operate to perform grayscale B-model processing and/or color processing. Grayscale B-model processing may include processing B-model RF signal data or IQ data pairs. For example, the grayscale B-model processing can enable the formation of an envelope of the received beam summing signal by computing the amount (I²+Q²)^{1/2}. The envelope can be subjected to additional B-model processing, such as logarithmic compression to form the display data. The display data can be converted to X-Y format for video display. Scan-converted frames can be mapped to grayscale for display. The B model frame is provided to the image buffer 250 and/or the display system 260. Color processing may include processing color-based RF signal data or IQ data pairs to form frames to cover the B-model frames being provided to image buffer 250 and/or display system 260. Grayscale and/or color processing may be adaptively adjusted based on user input (e.g., selections from the user input device 230), such as for enhancing the grayscale and/or color of a particular region.

The embodiments of the present application further provide a computer-readable program. The program, when executed, causes a computer to execute, in a medical imaging system, the method for generating information as described in any of the foregoing embodiments.

The embodiments of the present application further provide a storage medium storing a computer-readable program. The computer-readable program causes a computer to execute, in a medical imaging system, the method for generating information as described in any of the foregoing embodiments.

A non-transitory computer-readable medium stores a computer program. The computer program has at least one code segment, and the at least one code segment is executable by a machine (for example, a computer), so as to cause the machine to execute the method for generating information as described in any of the foregoing embodiments.

The above embodiments merely provide illustrative descriptions of the embodiments of the present application. However, the present application is not limited thereto, and suitable variations may be made on the basis of the above embodiments. For example, each of the above embodiments may be used independently, or one or more of the above embodiments may be combined.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the embodiments are clear according to the detailed description. Therefore, the appended claims are intended to cover all these features and advantages that fall within the true spirit and scope of these embodiments. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the embodiments.

1. A method for generating information, the method comprising:
obtaining electronic data of a first medical examination report;
analyzing content of the first medical examination report based on the electronic data; and
generating report information based on a result of the analysis, the report information being used to generate a second medical examination report.

2. The method according to claim 2 wherein,
the report information comprises an examination protocol of a medical examination to which the second medical examination report relates.

3. The method according to claim 2, wherein,
generating report information based on a result of the analysis comprises:
determining a type of a first medical examination in the first medical examination report based on a recognition result of text information of at least one information region in the first medical examination report, the type of the first medical examination being the same as a type of the medical examination to which the second medical examination report relates; and
generating the examination protocol based on the type of the first medical examination.

4. The method according to claim 3,
further comprising:
generating display control information based on the examination protocol, the display control information being used to control a display to display a user interface reflecting the examination protocol.

5. The method according to claim 4, wherein,
the user interface comprises:
a first icon for representing each examination step in the examination protocol, a display state of a first icon corresponding to a completion state of the examination step.

6. The method according to claim 5, wherein,
the user interface further comprises at least one of the following pieces of information:
a schematic diagram of an examination subject of at least one examination step;
a medical image obtained in the at least one examination step; and
an operation menu of the at least one examination step.

7. The method according to claim 2, wherein,
the report information further comprises an examination report template for generating the second medical examination report.

8. The method according to claim 7, wherein,
analyzing content of the first medical examination report based on the electronic data comprises:
recognizing content format information and layout information of a plurality of information regions in the first medical examination report;
recognizing text information in at least one of the information regions; and
associating each of the information regions in the first medical examination report with an examination report element based on recognition results of the content format information, the layout information, and the text information.

9. The method according to claim 8, further comprising:
generating an examination report element for the at least one of the information regions in the first medical examination report, and associating the information region with the generated examination report element.

10. The method according to claim 8, wherein,
generating report information based on a result of the analysis comprises:
generating the examination report template based on a result of the association.

11. The method according to claim 10, wherein,
in the examination report template, information in the at least one of the information regions in the first medical examination report is configured as predetermined information.

12. A medical apparatus, wherein the medical apparatus comprises:
a memory storing instructions; and
a processor configured to execute the instructions to:
obtain electronic data of a first medical examination report;
analyze content of the first medical examination report based on the electronic data; and
generate report information based on a result of the analysis, the report information being used to generate a second medical examination report.

13. A non-transitory computer-readable storage medium for storing a computer program, the computer program, when executed by a computer, causes the computer to:
obtain electronic data of a first medical examination report;
analyze content of the first medical examination report based on the electronic data; and
generate report information based on a result of the analysis, the report information being used to generate a second medical examination report.
